(19) United States
(12) Patent Application Publication
(10) Pub. No.: US 2024/0206979 A1
(43) Pub. Date: Jun. 27, 2024

(54) ENDOBRONCHIAL ULTRASOUND-GUIDED TRANSBRONCHIAL NEEDLE ASPIRATION (EBUS-TBNA) BRONCHOSCOPE

(71) Applicant: SoundCath, Inc., Canoga Park, CA (US)

(72) Inventors: Eric Stoppenhagen, Canoga Park, CA (US); Jesus Andres Lopez, Bloomington, CA (US)

(73) Assignee: SoundCath, Inc., Canoga Park, CA (US)

(21) Appl. No.: 18/146,432

(22) Filed: Dec. 26, 2022

(51) Int. Cl. A61B 34/20; A61B 8/00; A61B 8/08; A61B 8/12; A61B 10/02; B06B 1/06

(52) U.S. Cl. CPC A61B 34/20; A61B 8/085; A61B 8/12; A61B 8/445; A61B 8/4488; A61B 8/56; A61B 10/0283; B06B 1/0622; A61B 2010/045

(57) ABSTRACT

A pMUT guided ultrasound bronchoscope is disclosed. The pMUT guided ultrasound bronchoscope comprises an insertion tube having a proximal section and a distal section. A handle is connected to the proximal section, and a distal tip connected to the distal section of the insertion tube. The handle comprises a working channel entry port for inserting fluids and biopsy tools into a working channel extending through the insertion tube and exiting near the distal tip. A suction button is configured to control a valve disposed adjacent to the suction port for controlling suction when connected to a suction device.

### FIELD OF THE DISCLOSURE

The present disclosure relates generally to the field of ultrasonic catheters. More particularly, embodiments relate to a Piezoelectric Micromachined Ultrasonic Transducer (pMUT)-pulmonary endobronchial ultrasound transbronchial needle aspiration (EBUS-TBNA) or a TBNA bronchoscope for performing biopsy of bronchial lymph nodes.

### BACKGROUND OF THE DISCLOSURE

Lungs are the most common site where cancer may form, with 2.09 million new cases and 1.76 million deaths attributed to lung cancer worldwide in 2018. More than three quarters of lung cancer patients are not identified until after the disease has spread to distant or regional locations, at which point survival rates for the next five years range from 5.5% to 33.4%. It is so essential for the treatment and survival of a lung cancer patient to make a prompt diagnosis and perform an accurate staging. Such accurate and early diagnosis is difficult to achieve, and the associated costs are often exorbitant, particularly in areas with low resources. In these kinds of instances, diagnosis and staging may only be achieved through the performance of a lymph node (LN) biopsy and the subsequent classification of lymph nodes as benign or malignant.

Traditional diagnostic modalities for mediastinal and lung lesions have included non-invasive or minimally invasive approaches utilizing exfoliative cytology. Such traditional diagnostic modalities include procedures such as transbronchial needle aspiration (TBNA) or Wang needle biopsy, as well as computed tomography (CT)-guided transcutaneous fine needle aspiration. TBNA and Wang needle biopsies are both examples of diagnostic procedures. In addition to less invasive surgical techniques, such as mediastinoscopy and thoracoscopy, diagnostic tissue may also be obtained by these procedures. Endobronchial ultrasound-guided transbronchial needle aspiration (EBUS-TBNA), which is also known as EBUS-fine needle aspiration, is a relatively new method that collects samples from the lung and/or mediastinum using a needle with a fine gauge (22G or smaller). Such unique approach may move beyond just sampling of the pulmonary parenchyma because it incorporates the use of traditional bronchoscopy and ultrasound imaging, both of which have been in use for decades.

The efficacy of EBUS fine needle aspiration in sparing patients from more invasive procedures, such as EBUS for lung cancer staging in place of mediastinoscopy, and its capacity to hasten the proper treatment, have contributed to the procedure's rise in popularity. The bronchoscopic sampling procedure known as catheter biopsy is used to diagnose peripheral pulmonary lesions. Endobronchial ultrasonography, also known as EBUS, has found widespread application, which has resulted in new prospects for ultrasound-guided lymph node sampling. The diagnostic value of EBUS in determining whether cancer has spread to lymph nodes has demonstrated a high pooled sensitivity of 90% and a pooled specificity of 99%, according to a recent study.

Alternatively, a local intravenous anesthetic is used while performing EBUS-fine needle aspiration. An anesthesiologist needs to be present to oversee the monitoring of the patient's ECG, pulse oximetry, and blood pressure throughout the procedure. The EBUS-fine needle aspiration may be accomplished by inserting a flexible bronchoscope that contained an ultrasonic probe through the laryngeal mask. The bronchoscope is then guided through the trachea and the bronchial tree in the direction of the correct location of the mediastinum. The lesion is pierced with a needle that is inserted through the bronchial wall and into the bronchoscope. This allows for the tissue to be aspirated. It may be necessary to puncture a lymph node or mass three to four times to obtain a sufficient sample, and this procedure may be repeated on many lymph nodes during the same session. After that, the aspirates are smeared on slides and are concurrently delivered to the laboratory of the pathology department for further cytology. However, the visualization during the procedure is limited which increases the risk of puncturing different region of interest to obtain the biopsy sample.

Therefore, there is a need for an improved EBUS apparatus for bronchoscope biopsy with clear bronchoscopic visualization and ultrasound imaging techniques to locate the lymph nodes or masses of interest.

### SUMMARY OF THE DISCLOSURE

By way of introduction, the preferred embodiments described below include an easy-to-use Piezoelectric Micromachined Ultrasonic Transducer (pMUT) guided ultrasound bronchoscope is disclosed. The pMUT guided ultrasound bronchoscope comprises an insertion tube having a proximal section and a distal section. The insertion tube is inserted into a patient and directed towards a region of interest. A handle is connected to the proximal section, and a distal tip is connected to the distal section of the insertion tube. The handle comprises a working channel entry port for inserting fluids and biopsy tools into a working channel extending through the insertion tube and exiting near the distal tip. A suction button is provided and is configured to control a valve disposed adjacent to the suction port for controlling suction when connected to a suction device. Further, the handle comprises a flexion extension level to adjust location and orientation of the distal tip when directed toward the region of interest.

Further, the distal tip is disposed with an ultrasound transducer array having a transmit and receive circuitry. It may be noted that the distal tip is coated with a material to provide electrical isolation and transmission of ultrasound signals. The ultrasound transducer array includes a flexible interconnection between the transmit and receiver circuitry and the ultrasound transducer array. It may be noted that the ultrasonic transducer array corresponds to a micro-electromechanical (MEMS) based Piezoelectric Micromachined Ultrasonic Transducer (pMUT) or other MEMS based transducer. Further, the ultrasonic transducer array is coupled to an imaging device using a custom dongle, and the custom dongle is configured to communicate ultrasound transmit pulses and ultrasound receive waveforms. The ultrasonic transducer array comprises a plurality of transducer array elements having transducer cells of multiple diameters, to achieve a wide bandwidth. It may be noted that each of the plurality of transducer array elements is a linear phased array. The plurality of transducer array elements creates an individual focused beam.

According to another aspect of the invention, a Piezoelectric Micromachined Ultrasonic Transducer (pMUT) guided ultrasound bronchoscope is disclosed. The pMUT guided ultrasound bronchoscope comprises an insertion tube having a proximal section and a distal section. The insertion tube is inserted into a patient and directed towards a region of interest. A handle is connected to the proximal section, and a distal tip is connected to the distal section of the insertion tube. The handle comprises a working channel entry port for inserting fluids and biopsy tools into a working channel extending through the insertion tube and exiting near the distal tip. It may be noted that the biopsy tools include a biopsy needle extending from the working channel extending through the insertion tube and exiting near the distal tip. A suction button is provided and is configured to control a valve disposed adjacent to the suction port for controlling suction when connected to a suction device. Further, the handle comprises a flexion extension level to adjust location and orientation of the distal tip when directed toward the region of interest. Further, the pMUT guided ultrasound bronchoscope comprises an ultrasound transducer array having a transmit and receive circuitry disposed onto the distal tip towards the distal section of the insertion tube. The ultrasound transducer array is configured to communicate via the transmit and receive circuitry. Further, a biopsy needle located above the ultrasound transducer array and extending through the insertion tube and exiting near the distal tip.

### BRIEF DESCRIPTION OF THE DRAWINGS

The accompanying drawings illustrate various embodiments of systems, methods, and embodiments of various aspects of the disclosure. Any person of ordinary skill in the art will appreciate that the illustrated element boundaries (e.g., boxes, groups of boxes, or other shapes) in the figures represent one example of the various boundaries representative of the disclosed invention. It may be that in some examples one element may be designed as multiple elements or that multiple elements may be designed as one element. In other examples, an element shown as an internal component of one element may be implemented as an external component in another and vice versa. Furthermore, elements may not be drawn to scale. Non-limiting and non-exhaustive descriptions of the present disclosure are described with reference to the following drawings. The components in the figures are not necessarily to scale, emphasis instead being placed upon the illustrated principles.

Various embodiments will hereinafter be described in accordance with the appended drawings, which are provided to illustrate and not to limit the scope of the disclosure in any manner, wherein similar designations denote similar elements, and in which:

FIG. 1 illustrates a top view of an exemplary application of a Piezoelectric Micromachined Ultrasonic Transducer (pMUT) endobronchial ultrasound-guided transbronchial needle aspiration (EBUS-TBNA) bronchoscope for evaluation of lungs of a patient, according to an embodiment of the present disclosure;

FIG. 2 illustrates a side view of the pMUT-EBUS-TBNA bronchoscope delivered to a bronchos of the patient, according to an embodiment of the present disclosure;

FIG. 3 illustrates a schematic diagram of several locations of the pMUT-EBUS-TBNA bronchoscope for examination of bronchial lymph nodes, according to an embodiment of the present disclosure;

FIG. 4 illustrates a schematic diagram of the pMUT-EBUS-TBNA bronchoscope inserted into the bronchial lymph node, according to an embodiment of the present disclosure;

FIG. 5 illustrates a closeup schematic diagram of an insertion tube of the pMUT-EBUS-TBNA bronchoscope inserted into the bronchial lymph node, according to an embodiment of the present disclosure;

FIG. 6 illustrates a schematic diagram of an ultrasonic imaging system, according to an embodiment of the present disclosure;

FIG. 7 illustrates a sectional view of a distal end of the pMUT-EBUS-TBNA bronchoscope having the ultrasonic transducer array with a plurality of transducer array elements, according to an embodiment of the present disclosure;

FIG. 8 illustrates a side view of the pMUT-EBUS-TBNA bronchoscope, according to an embodiment of the present disclosure; and

FIG. 9 illustrates a side view of distal section of the insertion tube with a biopsy needle extending from a working channel exit port and the ultrasound transducer array, according to an embodiment of the present disclosure.

### DETAILED DESCRIPTION

The components of the embodiments as generally described and illustrated in the figures herein may be arranged and designed in a wide variety of different configurations. Thus, the following more detailed description of various embodiments, as represented in the figures, is not intended to limit the scope of the present disclosure but is merely representative of various embodiments. While various aspects of the embodiments are presented in drawings, the drawings are not necessarily drawn to scale unless specifically indicated.

Some embodiments of this disclosure, illustrating all its features, will now be discussed in detail. The words “comprising,” “having,” “containing,” and “including,” and other forms thereof, are intended to be equivalent in meaning and be open-ended in that an item or items following any one of these words is not meant to be an exhaustive listing of such item or items or meant to be limited to only the listed item or items.

It must also be noted that as used herein and in the appended claims, the singular forms “a,” “an,” and “the” include plural references unless the context dictates otherwise. Although any systems and methods similar or equivalent to those described herein may be used in the practice or testing of embodiments of the present disclosure, the preferred systems, and methods are now described. The terms “proximal” and “distal” are opposite directional terms. For example, the distal end of a device or component is the end of the component that is furthest from the practitioner during ordinary use. The proximal end refers to the opposite end, or the end nearest the practitioner during ordinary use.

Embodiments of the present disclosure will be described more fully hereinafter with reference to the accompanying drawings in which like numerals represent like elements throughout the several figures, and in which example embodiments are shown. Embodiments of the present disclosure may, however, be embodied in alternative forms and should not be construed as being limited to the embodiments set forth herein. The examples set forth herein are non-limiting examples and are merely examples among other possible examples.

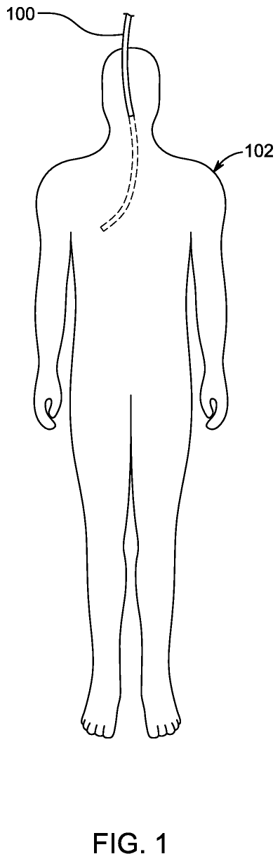

FIG. 1 illustrates a top view of an exemplary application of a Piezoelectric Micromachined Ultrasonic Transducer (pMUT) endobronchial ultrasound-guided transbronchial needle aspiration (EBUS-TBNA) bronchoscope 100 for evaluation of lungs of a patient 102, according to an embodiment of the present disclosure.

The pMUT-EBUS-TBNA bronchoscope 100 may be provided to perform evaluation of lungs by an operator. In one embodiment, the operator may be a pulmonologist or pulmonary interventionist or a surgeon or a doctor. The pMUT-EBUS-TBNA bronchoscope 100 is a technique to perform bronchoscopy or biopsy of lungs or lymph nodes. The pMUT-EBUS-TBNA bronchoscope 100 is a minimally invasive procedure that is performed for allowing sampling of mediastinal lymph nodes via fine-gauge aspiration needle (not shown) under direct sonographic visualization. The pMUT-EBUS-TBNA bronchoscope 100 has a low rate of morbidity and has demonstrated utility in the diagnosis of mediastinal lymphadenopathy secondary to malignancy, lymphoma, and sarcoidosis. The pMUT-EBUS-TBNA bronchoscope 100 allows the operator to obtain tissue or fluid samples from the lungs and surrounding lymph nodes without conventional surgery. The fluid samples may be used for diagnosing and staging lung cancer, detecting infections, and identifying inflammatory diseases that affect the lungs, such as sarcoidosis or cancers like lymphoma.

The fine-gauge aspiration needle is inserted via trachea towards the lymph nodes. The pMUT-EBUS-TBNA bronchoscope 100 may be performed needle aspiration on the lymph nodes using a bronchoscope (not shown) inserted through the mouth. The pMUT-EBUS-TBNA bronchoscope 100 may provide a real-time imaging of surface of the airways, blood vessels, lungs, and lymph nodes. It may be noted that an endoscope (not shown) may be fitted with an ultrasound processor (not shown) and a fine-gauge aspiration needle may be guided through the patient's trachea. In one embodiment, the pMUT-EBUS-TBNA bronchoscope 100 may provide improved images which allow the operator to easily view difficult-to-reach areas or region of interest and to access more, and smaller, lymph nodes for biopsy with the aspiration needle than through conventional mediastinoscopy.

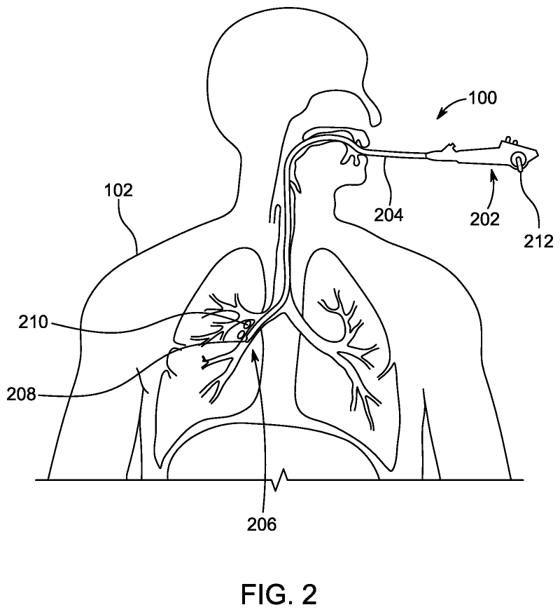

FIG. 2 illustrates a side view of the pMUT-EBUS-TBNA bronchoscope 100 delivered to a bronchos of the patient 102, according to an embodiment of the present disclosure.

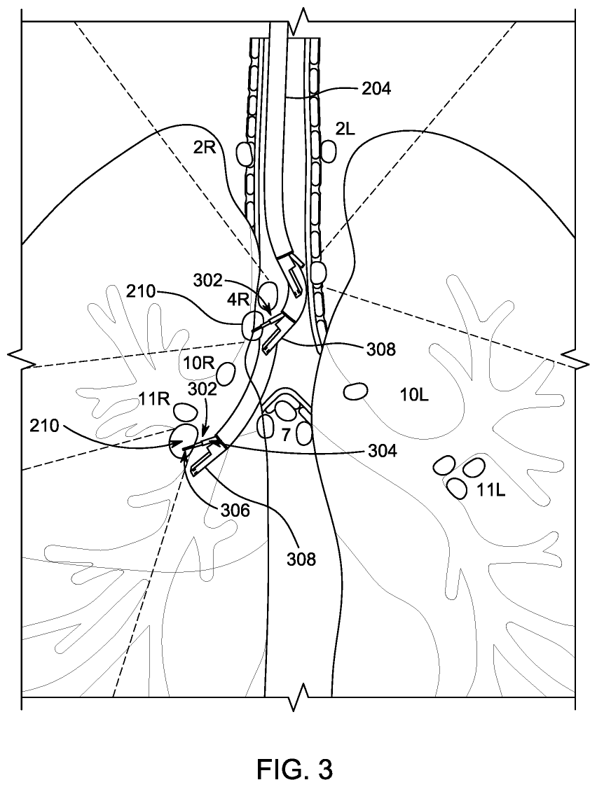

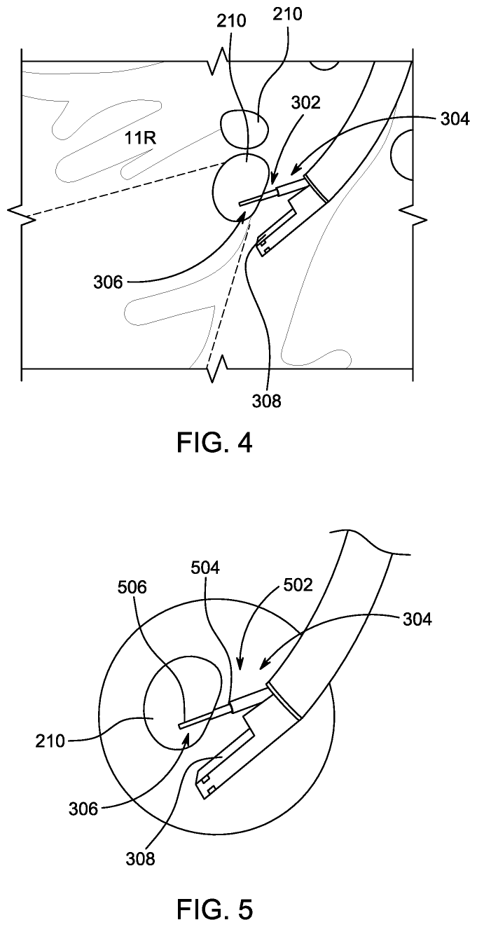

The pMUT-EBUS-TBNA bronchoscope 100 may comprise a handle 202 connected to an insertion tube (not shown) via a flexible shaft 204. The pMUT-EBUS-TBNA bronchoscope 100 may be positioned at an inclined angle to face or mouth of the patient 102 when the insertion tube is inserted into trachea via mouth. In one embodiment, the flexible shaft 204 may be made from a material selected from a group of materials such as, but are not limited to, polymer, carbon fiber, aluminum, or other elastic materials. The insertion tube may comprise a proximal section and a distal section. The insertion tube may be inserted into the trachea of the patient and directed towards a region of interest 206, guided by the handle 202. The pMUT-EBUS-TBNA bronchoscope 100 may comprise a distal tip 208 disposed towards the distal section of the insertion tube. It may be noted that the distal tip 208 of the insertion tube may be directed towards the region of interest. Further, the distal tip 208 of the insertion tube may be positioned in proximity to one or more bronchial lymph nodes 210. The one or more bronchial lymph nodes 210 may be detected using an ultrasound imaging technique. Further, the distal tip 208 of the insertion tube may be provided with a biopsy needle (not shown) to extract tissue samples from the one or more bronchial lymph nodes 210. The insertion tube is described later in detail in conjunction with FIGS. 3-4 of the present disclosure.

Further, the pMUT-EBUS-TBNA bronchoscope 100 may comprise a flexion extension lever 212 positioned towards the handle 202. The flexion extension lever 212 may be configured to adjust location and orientation of the distal tip 208 when directed toward the region of interest. In one embodiment, the handle 202 may be manipulated by the operator to adjust the position of the distal tip 208 along with the biopsy needle proximal towards the one or more bronchial lymph nodes 210. In one exemplary embodiment, the operator uses a left hand to hold the handle 202 of the pMUT-EBUS-TBNA bronchoscope 100 and inserts the flexible shaft 204 into the trachea via the mouth of the patient 102, then using a right hand manipulates the flexion extension lever 212 when the distal tip 208 is in proximity to the one or more bronchial lymph nodes 210.

FIGS. 3-4 illustrate a schematic diagram of several locations of the pMUT-EBUS-TBNA bronchoscope 100 for examination of the one or more bronchial lymph nodes 210, according to an embodiment of the present disclosure.

The pMUT-EBUS-TBNA bronchoscope 100 may comprise the flexible shaft 204 inserted via the trachea towards the lungs or the one or more bronchial lymph nodes 210. The flexible shaft 204 may comprise one or more detachable sections (not shown). The one or more detachable sections may be assembled according to the one or more bronchial lymph nodes 210 on different lobes of lungs of the patient 102. For example, 5 detachable sections of the flexible shaft 204 are used when a right lower lobe (RLL) of the lung of the patient 102 is accessed for lymph node biopsy.

Further, the pMUT-EBUS-TBNA bronchoscope 100 may comprise one or more insertion tubes 302 disposed towards the distal tip 208. Each of the one or more insertion tubes 302 may comprise a proximal section 304 and a distal section 306. The proximal section 304 of each of the one or more insertion tubes 302 may be coupled to the flexible shaft 204 towards the one or more detectable sections. In one embodiment, the one or more insertion tubes 302 may be provided to access multiple biopsies or multiple lung samples from the one or more bronchial lymph nodes 210. Therefore, the pMUT-EBUS-TBNA bronchoscope 100 may be employed for accessing multiple biopsies from the one or more bronchial lymph nodes 210 located at different locations or lobes of the lungs of the patient 102. Further, the one or more insertion tubes 302 may be provided with one or more biopsy needles (not shown) to extract the biopsy sample from the one or more bronchial lymph nodes 210. It may be noted that the one or more biopsy needles may be disposed towards the distal section 306 of each of the one or more insertion tubes 302.

Further, the flexible shaft 204 may comprise one or more spherical joints (not shown) to couple the one or more detachable sections. The one or more spherical joints may be coupled at different lengths to provide a diversion for the flexible shaft 204 when inserted inside the trachea of the patient 102. For example, a flexible shaft of an over length of 30 centimeters (cm) is having five sections with four detachable sections and therefore, four spherical joints. In one embodiment, the one or more spherical joints may include coupling joints, such as, but not limited to, ball joints, bearing joints, spherical joints, and U-joints. In another embodiment, the one or more spherical joints may be made from a material selected from a group of materials of carbon fiber, stainless steel, titanium, and other non-conductive and corrosion free materials.

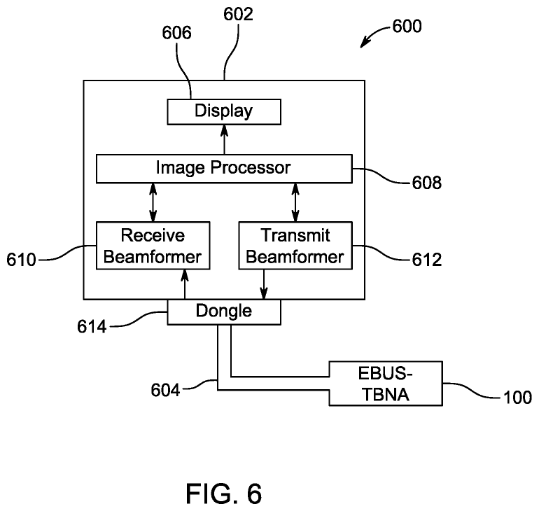

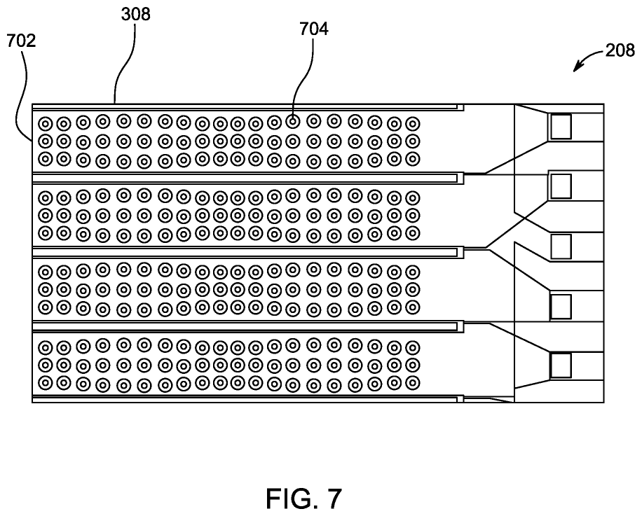

Further, the pMUT-EBUS-TBNA bronchoscope 100 may comprise one or more ultrasonic transducer arrays 308 disposed towards the distal section 306 of each of the one or more insertion tubes 302. The one or more ultrasonic transducer arrays 308 may be integrated onto the distal tip 208 of the pMUT-EBUS-TBNA bronchoscope 100. In one embodiment, the one or more ultrasonic transducer arrays 308 may provide mapping and imaging details of the one or more bronchial lymph nodes 210 when the one or more insertion tubes 302 and the flexible shaft 204 is inserted inside the trachea of the patent 202. The one or more ultrasonic transducer arrays 308 may provide details related to target lymph nodes where the biopsy samples need to be extracted. The one or more ultrasonic transducer arrays 308 is described in detail in conjunction with FIGS. 6-7 of the present disclosure.

FIG. 5 illustrates a closeup schematic diagram of an insertion tube 502 of the pMUT-EBUS-TBNA bronchoscope 100 inserted into a bronchial lymph node of the one or more bronchial lymph nodes 210, according to an embodiment of the present disclosure.

The insertion tube 502 of the pMUT-EBUS-TBNA bronchoscope 100 may be inserted into the bronchial lymph node of the one or more bronchial lymph nodes 210 to extract biopsy sample. The insertion tube 502 may comprise a working channel exit port 504 positioned towards the proximal section 304 of the insertion tube 502. The working channel exit port 504 may be provided with a biopsy needle 506 extending towards the distal section 306 of the insertion tube 502. The biopsy needle 506 may be manipulated by the handle 202 to extract sample from the bronchial lymph node of the one or more bronchial lymph nodes 210.

Further, the pMUT-EBUS-TBNA bronchoscope 100 may comprise the one or more ultrasonic transducer arrays 308 disposed onto the distal tip 208 of the flexible shaft 204. The one or more ultrasonic transducer arrays 308 may transmit ultrasound signals to the bronchial lymph node of the one or more bronchial lymph nodes 210 and may receive echo signals, which may be transmitted to an external system for image construction and mapping of the bronchial lymph node of the one or more bronchial lymph nodes 210. The construction and arrangement of the one or more ultrasonic transducer arrays 308 is described in conjunction with FIGS. 6-7.

FIG. 6 illustrates a schematic diagram of an ultrasonic imaging system 600, according to an embodiment of the present disclosure.

The ultrasonic imaging system 600 may utilize the one or more ultrasonic transducer arrays 308 interconnected using matched flexible circuits. In one embodiment, the one or more ultrasonic transducer arrays 308 may be a microelectromechanical (MEMS) transducer array defined as piezoelectric micro-machined ultrasound transducer (pMUT) or other types of MEMS transducers. It may be noted that the ultrasonic imaging system 600 uses high-density flexible circuits that may enable highly repeatable and stable transmission and return signals. Further, the high-density flexible circuit transmission lines may transmit electrical energy from one end to another distal end of the ultrasonic imaging system 600.

The ultrasonic imaging system 600 may comprise an imaging device 602 linked to the pMUT-EBUS-TBNA bronchoscope 100 via a communication channel 604. The imaging device 602 may comprise a display 606, an image processor 608, a receive beamformer 610, a transmit beamformer 612 and a dongle 614. The pMUT-EBUS-TBNA bronchoscope 100 may be disposed within the one or more bronchial lymph nodes 210 of the patient 102 and the imaging device 602 may receive at least one signal from the one or more ultrasonic transducer arrays 308. The at least one signal may be communicated to the imaging device 602 via an electronic flex cable (not shown) connected to the dongle 614.

The image processor 608 may be configured to generate a two-dimensional (2D) image according to data received from the pMUT-EBUS-TBNA bronchoscope 100. In one embodiment, the image processor 608 may be configured to receive a focused signal from the receive beamformer 610. The image processor 608 may render the data to construct an image or sequence of images. In one embodiment, the image may be three-dimensional (3D) representation, such as a two-dimensional image rendered from a user or a processor selected viewing direction. In one embodiment, the image processor 608 may be a detector, filter, processor, application-specific integrated circuit, field-programmable gate array, digital signal processor, control processor, scan converter, three-dimensional image processor, graphics processing unit, analog circuit, digital circuit, or combinations thereof. The image processor 608 may receive beamformed data and may generate images, to display on the display 606. It may be noted that the generated images are associated with a two-dimensional (2D) scan. Alternatively, the generated images may be three-dimensional (3D) representations.

The image processor 608 may be programmed for hardware accelerated two-dimensional re-constructions. The image processor 608 may store processed data of the at least one signal and a sequence of images in a memory. In one embodiment, the memory may be a non-transitory computer-readable storage media. The instructions for implementing the processes, methods and/or techniques discussed herein are provided on the computer-readable storage media or memories, such as a cache, buffer, RAM, removable media, hard drive, or other computer-readable storage media. Non-transitory computer-readable storage media include various types of volatile and non-volatile storage media. The functions, acts, or tasks illustrated in the figures or described herein are executed in response to one or more sets of instructions stored in or on a computer readable storage media. The functions, acts, or tasks are independent of the particular type of instruction sets, storage media, processor, or processing strategy and may be performed by software, hardware, integrated circuits, firmware, micro code, and the like, operating alone or in combination.

The pMUT-EBUS-TBNA bronchoscope 100 may be in electronic communication with the imaging device 602 for transmission and receiving of ultrasound signals to and from the one or more bronchial lymph nodes 210. In one embodiment, the pMUT-EBUS-TBNA bronchoscope 100 may be configured to visualize standard views of the one or more bronchial lymph nodes 210 of the lungs of the patient 102, such as in a standard version, right lobes and left lobes may be visualized.

FIG. 7 illustrates a sectional view of the distal tip 208 of the pMUT-EBUS-TBNA bronchoscope 100 having the one or more ultrasonic transducer arrays 308 with a plurality of transducer array elements 702, according to an embodiment of the present disclosure.

The one or more ultrasonic transducer arrays 308 may comprise the plurality of transducer array elements 702, arranged towards the distal tip 208 of the pMUT-EBUS-TBNA bronchoscope 100. Further, each of the plurality of transducer array elements 702 may have a plurality of individual transducer cells 704 arranged in a manner to provide a wide bandwidth of the individual focused beam. In one embodiment, the one or more ultrasonic transducer arrays 308 may be constructed from a pMUT based array containing individual elements of different diameters. In one embodiment, to achieve wider bandwidth with pMUT based arrays, multiple diameters of pMUT cells may be integrated into one element. It may be noted that by arranging pre-shaped pMUT cells with different diameters, a broader bandwidth may be realized through the complex interaction between the individual pMUT elements. In one embodiment, the pMUT cells of multiple diameters may achieve a bandwidth of greater than 55%. For example, in 3 elements, there are 5 different dome diameters, and each array is of a different size, such as 300 μm.

Further, the one or more ultrasonic transducer arrays 308 may correspond to pMUT and the plurality of transducer array elements 702 may correspond to a plurality of pMUT elements. In one embodiment, the plurality of transducer array elements 702 may be directed to transmit and receive, the ultrasound beams having the bandwidth including the predetermined fundamental mode vibration of each of the plurality of transducer array elements 702, such that a single transducer array element may transmit and receive multiple fundamental mode vibrations simultaneously. Further, the electronic flex cable inside the flexible shaft 204 receives the at least one signal from the plurality of transducer array elements 702. It may be noted that the at least one signal may correspond to the at least one ultrasound beam. The at least one signal may be transmitted to the imaging device 602 for further processing in the image processor 608. The image processor 608 may construct at least one image of the one or more bronchial lymph nodes 210. It may be noted that the plurality of transducer array elements 702 may be used to create the individual focused beam. In one embodiment the plurality of transducer array elements 702 are arranged in a linear fashion. In a second embodiment the plurality of transducer array elements 702 are arranged in cylinder fashion.

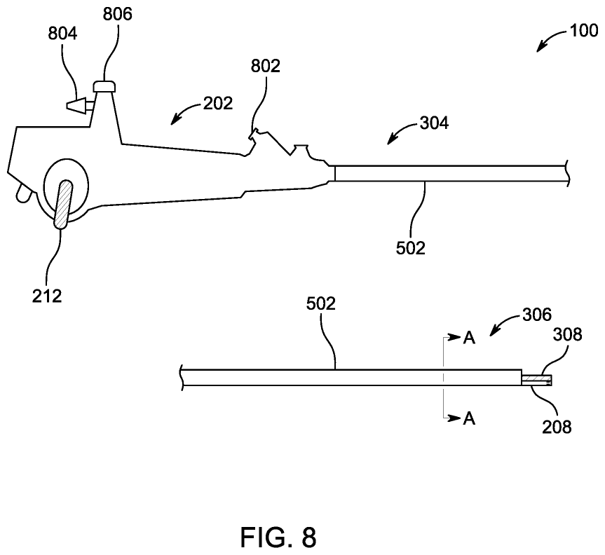

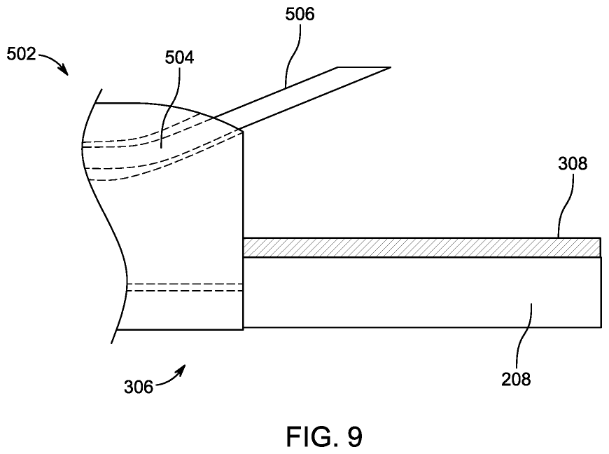

FIG. 8 illustrates a side view of the pMUT-EBUS-TBNA bronchoscope 100, according to an embodiment of the present disclosure.

The pMUT-EBUS-TBNA bronchoscope 100 may comprise the handle 202, the insertion tube 502 with the proximal section 304 and the distal section 306. The proximal section 304 of the insertion tube 502 may be connected to the handle 202 via the flexible shaft 204. The distal section 306 is positioned between the distal tip 208 and the proximal section 304. Further, handle 202 may comprise a working channel entry port 802, a suction port 804 and a suction button 806. The working channel entry port 802 may be integrated on the handle 202 towards the flexible shaft 204. In one embodiment, the working channel entry port 802 may be employed for inserting fluids and/or tools into a working channel (not shown) extending through the insertion tube 502 and exiting near the distal tip 208.

Further, the suction button 806 may be configured to control a valve adjacent to the suction port 804 for the controlling suction when the suction port 804 is connected to a suction device (not shown). In one embodiment, the suction button 806 is pressed or the suction port 804 is activated to suck blockage, such as, mucus, blood, etc., when the insertion tube 502 is inserted inside the trachea of the patient 102.

As discussed above, the handle 202 may comprise the flexion extension lever 212. The flexion extension lever 212 may be configured to adjust location and orientation of the distal tip 208 when directed toward the one or more bronchial lymph nodes 210. Further, the distal tip 208 of the pMUT-EBUS-TBNA bronchoscope 100 may be provided with the one or more ultrasonic transducer arrays 308. The one or more ultrasonic transducer arrays 308 may transmit ultrasound signals continuously when the handle 202 manipulates the insertion tube 502 to generate different images of the surrounding lymph nodes.

FIG. 9 illustrates a side view of distal section 306 of the insertion tube 502 with the biopsy needle 506 extending from the working channel exit port 504 and the ultrasound transducer array 308, according to an embodiment of the present disclosure.

The biopsy needle 506 may extend from the working channel exit port 504 towards the bronchial lymph node of the one or more bronchial lymph nodes 210. The one or more ultrasonic transducer arrays 308 disposed over the distal tip 208 may be configured to transmit ultrasound signals to the surrounding lymph nodes and the imaging device 602 may receive the at least one signal to generate an image of the surrounding lymph nodes. A doctor/surgeon may the perform diagnosis in real-time and extract the biopsy sample from an infected lymph node using the biopsy needle 506.

In one embodiment, the pMUT-EBUS-TBNA bronchoscope 100 may be designed to enable delivery of the distal tip 208 to the bronchial lymph node of the one or more bronchial lymph nodes 210 or bronchi of the patient 102. In another embodiment, the pMUT-EBUS-TBNA bronchoscope 100 is designed with good push-ability, torque-ability, and steer-ability to enable an operator to easily manipulate distal tip 208 to a desired location and orientation.

In one embodiment, transducer array elements may have one or more different shapes. As used herein, the term “proximal and distal sections” refers to a tube or shaft, on which one or more transducer elements are disposed. The insertion tube 502, and the flexible shaft 204 are not limited to, size, or shape, and may be configured to be in expanded and unexpanded or compact states. As used herein, the term “proximal and distal sections” refer to a wire-like shaft capable of interfacing with transducer array elements. Further, the distal tip 208 is not limited to any size or measurement.

Other embodiments of the invention will be apparent to those skilled in the art from consideration of the specification and practice of the invention disclosed herein. It is intended that the specification and examples be considered as exemplary only, with a true scope and spirit of the invention being indicated by the following claims. In addition, where this application has listed the steps of a method or procedure in a specific order, it may be possible, or even expedient in certain circumstances, to change the order in which some steps are performed, and it is intended that the particular steps of the method or procedure claim set forth here below not be construed as being order-specific unless Such order specificity is expressly stated in the claim.

What is claimed is:

1. A piezoelectric Micromachined Ultrasonic Transducer (pMUT) guided ultrasound bronchoscope, comprising:
an insertion tube having a proximal section and a distal section; and
a handle connected to the proximal section, and a distal tip connected to the distal section of the insertion tube, wherein the handle comprises:
a working channel entry port for inserting fluids and biopsy tools into a working channel extending through the insertion tube and exiting near the distal tip; and
a suction button configured to control a valve disposed adjacent to the suction port for controlling suction when connected to a suction device.

2. The pMUT guided ultrasound bronchoscope of claim 1, wherein the insertion tube is inserted into a patient and directed towards a region of interest.

3. The pMUT guided ultrasound bronchoscope of claim 1, wherein the handle comprises a flexion extension lever to adjust location and orientation of the distal tip when directed toward a region of interest.

4. The pMUT guided ultrasound bronchoscope of claim 1, wherein the biopsy tools include a biopsy needle extending through the insertion tube and exiting near the distal tip.

5. The pMUT guided ultrasound bronchoscope of claim 1, wherein the distal tip is disposed within an ultrasound transducer array having a transmit and receive circuitry.

6. The pMUT guided ultrasound bronchoscope of claim 5, wherein the ultrasound transducer array includes a flexible interconnection between the transmit and receiver circuitry and the ultrasound transducer array.

7. The pMUT guided ultrasound bronchoscope of claim 5, wherein the ultrasonic transducer array corresponds to a micro-electromechanical (MEMS) based Piezoelectric Micromachined Ultrasonic Transducer (pMUT).

8. The pMUT guided ultrasound bronchoscope of claim 1, wherein the distal tip is coated with a material to provide electrical isolation and transmission of ultrasound signals.

9. The pMUT guided ultrasound bronchoscope of claim 5, wherein the ultrasonic transducer array is coupled to an imaging device using a custom dongle, and the custom dongle is configured to communicate ultrasound transmit pulses and ultrasound receive waveforms.

10. The pMUT guided ultrasound bronchoscope of claim 1, wherein the ultrasonic transducer array comprises a plurality of transducer array elements having transducer cells of multiple diameters, to achieve a wide bandwidth.

11. The pMUT guided ultrasound bronchoscope of claim 10, wherein each of the plurality of transducer array elements is a linear phased array, and the plurality of transducer array elements creates an individual focused beam.

12. A piezoelectric Micromachined Ultrasonic Transducer (pMUT) guided ultrasound bronchoscope, comprising:
an insertion tube having a proximal section and a distal section;
a handle is connected to the proximal section, and a distal tip connected to the distal section of the insertion tube, wherein the handle comprises:
a working channel entry port for inserting fluids and biopsy tools into a working channel extending through the insertion tube and exiting near the distal tip; and
a suction button configured to control a valve disposed adjacent to the suction port for controlling suction when connected to a suction device; and
an ultrasound transducer array having a transmit and receive circuitry disposed onto the distal tip towards the distal section of the insertion tube, wherein the ultrasound transducer array is configured to communicate via the transmit and receive circuitry.

13. The pMUT guided ultrasound bronchoscope of claim 12, wherein the insertion tube is inserted into a patient and directed towards a region of interest.

14. The pMUT guided ultrasound bronchoscope of claim 12, wherein a biopsy needle located above the ultrasound transducer array and extending through the insertion tube and exiting near the distal tip.

15. The pMUT guided ultrasound bronchoscope of claim 12, wherein the ultrasound transducer array includes a flexible interconnection between the transmit and receiver circuitry and the ultrasound transducer array.

16. The pMUT guided ultrasound bronchoscope of claim 12, wherein the ultrasonic transducer array corresponds to a micro-electromechanical (MEMS) based Piezoelectric Micromachined Ultrasonic Transducer (pMUT).

17. The pMUT guided ultrasound bronchoscope of claim 12, wherein the distal tip is coated with a material to provide electrical isolation and transmission of ultrasound signals.

18. The pMUT guided ultrasound bronchoscope of claim 12, wherein the ultrasonic transducer array is coupled to an imaging device using a custom dongle, and the custom dongle is configured to communicate ultrasound transmit pulses and ultrasound receive waveforms.

19. The pMUT guided ultrasound bronchoscope of claim 12, wherein the ultrasonic transducer array comprises a plurality of transducer array elements having transducer cells of multiple diameters, to achieve a wide bandwidth.

20. The pMUT guided ultrasound bronchoscope of claim 19, wherein each of the plurality of transducer array elements is a linear phased array, and the plurality of transducer array elements creates an individual focused beam.
